(19) United States
(12) Patent Application Publication
(10) Pub. No.: US 2016/0067520 A1
(43) Pub. Date: Mar. 10, 2016

(54) Photodynamic Therapy

(71) Applicant: AMBICARE HEALTH LIMITED, Livingston (GB)

(72) Inventor: Andrew MCNEILL, Livingston (GB)

(21) Appl. No.: 14/888,933

(22) PCT Filed: Apr. 7, 2014
(86) PCT No.: PCT/GB2014/051078

§ 371 Date: Nov. 3, 2015

(30) Foreign Application Priority Data
May 3, 2013 (GB) 1308039.5

(51) Int. Cl. A61N 5/06

(52) U.S. Cl. CPC A61N 5/062; A61N 5/0624; A61N 5/0616; A61N 2005/0661; A61N 2005/0662; A61N 2005/0659; A61N 2005/0653; A61N 2005/0654

(57) ABSTRACT

There is herein described apparatus and a method for performing Photodynamic Therapy (PDT) and apparatus for performing PDT. More particularly, there is herein described a method of using PDT and associated apparatus over a prolonged period for the treatment of a range of therapies such as wound healing, cancer treatment (including both skin and internal body cancer) and psoriasis.

### FIELD OF THE INVENTION

The present invention relates to Photodynamic Therapy (PDT) and apparatus for performing PDT. More particularly, the present invention relates to using PDT and associated apparatus over a prolonged period for the treatment of a range of therapies such as wound healing, cancer treatment (including both skin and internal body cancer) and psoriasis.

### BACKGROUND OF THE INVENTION

Photodynamic Therapy (PDT) is usually a two-step process and consists of:

1. Application of a photosensitiser to a treatment site; and

2. Subsequent illumination of the treatment site with light.

The light is used to activate a photosensitiser which is then be used to treat the treatment site. For example, PDT is used to treat a wide range of indications such as non-melanoma skin cancer (NMSC), wound healing, scar reduction, acne and the like.

We also refer to WO 03/043697 which relates to a prior art photodynamic therapy treatment and apparatus. WO 03/043697 is incorporated herein by reference.

PDT may be used to treat wounds (either chronic or acute) and this includes chronic leg ulcers and diabetic foot ulcers. PDT has also been shown to promote wound healing via a number of factors including reduction of bacterial load and promotion of growth factors. The presence of bacteria within the wound is known to be one of the most significant factors in reducing wound healing. However, one of the problems associated with existing PDT processes is that although current PDT processes are very efficient in killing bacteria within the wound, once the light source is switched off, the treatment is effectively stopped, and the bacteria are free to return and hamper the wound healing process.

It is an object of at least one aspect of the present invention to obviate or mitigate at least one or more of the aforementioned problems.

It is a yet further object of at least one aspect of the present invention to provide apparatus capable of providing an improved PDT capable of providing the treatment of a range of therapies such as wound healing, cancer treatment (including both skin and internal body cancer) and psoriasis.

It is a yet further object of at least one aspect of the present invention to provide a method of improved PDT capable of providing the treatment of a range of therapies such as wound healing, cancer treatment (including both skin and internal body cancer) and psoriasis.

### SUMMARY OF THE INVENTION

According to a first aspect of the present invention there is provided apparatus capable of performing photodynamic therapy (PDT), said apparatus comprising:

a first radiation source capable of irradiating a photosensitiser on a patient to be treated in a first radiation stage with a first wavelength over a short period of time ranging from about one minute to about three hours, wherein the photosensitiser is capable of performing medical treatment;

a second radiation source capable of irradiating the surface of the patient to be treated in a second radiation stage with a second wavelength over a longer period of time ranging from about six hours to about one month.

The present invention therefore relates to a first and second illumination stage which is intended to provide medical treatment such as wound healing, cancer treatment (including both skin and internal body cancer) and psoriasis.

The apparatus of the present invention may comprise means for applying a photosensitiser to a surface of a patient to be treated. Alternatively, the photosensitiser may be applied may be applied using a separate device or means.

The first and second wavelengths may be the same or different.

The treatment area on the patient may be on the surface or internally in a patient.

Typically, the first treatment stage may comprise applying a photosensitiser and then applying irradiation to the photosensitiser. The first radiation source may emit light which is capable of providing the irradiation for the first stage and may emit light over a desired wavelength. Typically, the radiation source may operate within a range of about 300-3000 nm, about 300-1500 nm, about 300-800 nm or about 400-700 nm. In particular embodiments the radiation source may operate in the visible region of the electromagnetic spectrum. Alternatively, the radiation source may operate in the ultraviolet or infrared wavelength regions of the electromagnetic spectrum.

In particular embodiments the first radiation source may emit radiation in range of between about 1 minute to about three hours or preferably about 10 minutes to about 1 hour. The emitted radiation may be constant or pulsed.

The first radiation source may have an optical power density of about 20-200 mW/cm². The radiation source during operation may operate at a substantially constant power or alternatively may be varied over a range of powers.

The first radiation stage may reduce and/or eliminate any bacteria in a wound or be used to treat cancer (including both skin and internal body cancer) and psoriasis.

The photosensitiser may be a photochemical and/or photopharmaceutical preparation. The photochemical and/or a photopharmaceutical preparation may be present in the form of, for example, a gel, ointment, cream or gauze soaked in a photodynamic therapy solution. Typical drugs and/or chemicals used in the present invention include but are not limited to Aminolevulinic Acid (ALA), Metvix, Methylene blue, Phenothiazinium compounds, Levulan and the like. The photosensitiser can be any of the following: photosensitisers which are applied and are converted/metabolised into a light sensitive compound; photosensiters which are applied that are already light sensitive; and photosensiters that are already light sensitive that preferentially accumulate in another medium such as bacteria.

As indicated above prior art treatment processes then stop the irradiation with the result that bacteria and the like may start to grow again leading to the wound becoming infected or cease the treatment of the cancer or psoriasis. This has a serious impact on the healing of the wound and the treatment of the cancer or psoriasis.

The second radiation stage irradiates the surface of the patient to be treated in a second radiation stage over a longer period of time ranging from about six hours to three months. Preferably, the second radiation source may emit radiation over a period of about 1 week to about 1 month. During this period the wound is kept free or substantially free of bacteria which facilitates the healing of the wound.

The second radiation stage may be performed with an ambulatory radiation source i.e. a portable radiation source.

Typically, the radiation in the second radiation source may be emitted in a non-constant or continuous manner and over a lower power than used in the first radiation stage. The treatment process therefore also comprises a second stage that consists of a relatively low level, long term PDT treatment that is carried out immediately or shortly following the first stage. The optical power that is used in the second stage may be about 0.1-20 mW/cm².

The photosensitiser used in the second stage is preferably different to that used in the first stage. Typically, the photosensitiser used in the second stage also has a lower concentration than that used in the first stage and, for example, may have a concentration of about 1-20% that of the concentration used in the first stage. In particular embodiments, the photosensitiser used in the first and second stages is different. In these types of embodiments the wavelength of radiation used in the first and second stages will also be different.

In particular embodiments, the first and second radiation sources may, for example, be any suitable form of diode such as organic light-emitting diode or an inorganic light-emitting diode incorporated into, for example, a diffuser. Alternatively, the light source may be a fluorescent light source such as a fluorescent lamp. The fluorescent light source may be compact in shape. For example, this includes technology developed from back-lit display technology, LEDs and waveguides and diffusers.

In the case of wound healing, the first treatment stage therefore kills large quantities of bacteria and the low level second stage prevent the bacteria from returning, maintaining them at low levels.

In particular embodiments, in the second irradiation stage the photosensitiser and light source may be administered simultaneously. Alternatively, the photosensitiser and illumination from the light source may be administered in sequence i.e. one after the other. In this situation, the photosensitiser application time would be the same as a typical PDT which is about 1-3 hours.

The photosensitiser may be applied at several times within the overall second irradiation stage. For example, this application may take place once a day for a month.

The extended duration of the second irradiation stage may require an ambulatory device i.e. a mobile device to be worn by a person so that they no longer need to be kept as an in-house patient in a hospital.

In particular embodiments, it is preferred to administer the photosensitiser and light radiation simultaneously in at least one or both the first and second radiation stages. This may be achieved by a drug delivery mechanism which is capable of releasing photosensitiser in a controlled manner over the duration of the second stage. This drug delivery mechanism may be a transparent drug delivery layer or some form of iontophoretic drug delivery layer.

The long duration of the second irradiation stage may require that a light source in the second radiation step is substantially off for most of the second stage. In this configuration, the light source may blip on for a short time such as for about 1 to 1000 s. For example, this may be used to maintain low levels of bacteria. This ultra-low level light treatment may require a correspondingly low concentration of photosensitiser such as about 1-20% of the concentration of the photosensitiser used in the first stage.

According to a second aspect of the present invention there is provided a method capable of performing photodynamic therapy (PDT), said method comprising:

providing a first radiation source capable of irradiating the photosensitiser on a patient to be treated in a first radiation stage with a first wavelength over a short period of time ranging from about one minute to about three hours, wherein the photosensitiser is capable of performing medical treatment; and

providing a second radiation source capable of irradiating the surface of the patient to be treated in a second radiation stage with a second wavelength over a longer period of time ranging from about six hours to about one month.

The apparatus used may be as defined in the first aspect.

### BRIEF DESCRIPTION

Generally speaking, the present invention therefore resides in the provision of a PDT treatment apparatus and method wherein there is a first irradiation stage which has a relatively high power over a relatively short period of time and thereafter a second irradiation stage over a much longer period of time but at a lower power.

In a particular embodiment of the present invention a photosensitiser such as Aminolevulinic Acid (ALA), Metvix, Methylene blue, Phenothiazinium compounds, Levulan and the like is applied to a patient. An effecttive amount of photosensitiser is applied. The photosensitiser may be applied on any position on a person's body including any internal area.

The applied photosensitiser is then subjected to irradiation from a light source in a first irradiation stage operating at about 300-800 nm (or preferably 400-750 nm) with a power of about 20-200 mw/cm² for about 10 minutes to about three hours. The radiation during this period is mostly switched on.

Then the patient is fitted with an ambulatory light emitting device which is capable of applying radiation in a second irradiation stage. The light source in the second irradiation stage operates at about 300-800 nm (or preferably 400-750 nm) with a power of about 0.1-20 mw/cm² over a period of about six hours to about four weeks. The radiation during this period is mostly switched off with the radiation being switched on at pre-determined intervals to ensure that bacteria is prevented from forming. Low levels of photosensitiser are used in the second stage such as about 1-20% of the concentration of the photosensitiser used in the first stage.

Whilst specific embodiments of the present invention have been described above, it will be appreciated that departures from the described embodiments may still fall within the scope of the present invention. For example, any suitable type of photosensitiser may be used. Furthermore, any suitable wavelength of radiation may be used.

1. Apparatus for a photodynamic therapy (PDT) treatment process capable of providing wound healing, said apparatus comprising:
a first radiation source capable of irradiating the photosensitiser on a patient to be treated in a first radiation stage with a first wavelength over a short period of time ranging from about one minute to about three hours, wherein the photosensitiser is capable of performing medical treatment;
a second radiation source capable of irradiating the surface of the patient to be treated in a second radiation stage with a second wavelength over a longer period of time ranging from about six hours to about one month.

2-29. (canceled)

30. Apparatus for a photodynamic therapy (PDT) treatment according to claim 1, wherein the second radiation stage is capable of being performed with an ambulatory radiation source.

31. Apparatus for a photodynamic therapy (PDT) treatment according to claim 1, wherein the first and second radiation stages eliminate and/or reduce bacteria content over a sustained period of time which facilitates wound healing.

32. Apparatus for a photodynamic therapy (PDT) treatment according to a claim 1, wherein the first and second radiation stages are used to treat cancer (including both skin and internal body cancer) and psoriasis.

33. Apparatus for a photodynamic therapy (PDT) treatment according to claim 1, wherein the first treatment stage comprises apparatus capable of applying a photosensitiser and then applying irradiation to the photosensitiser.

34. Apparatus for a photodynamic therapy (PDT) treatment according to claim 1, wherein the first radiation source emits light which is capable of providing the irradiation for the first stage and emits light over a desired wavelength; and
wherein the first and second radiation sources operate within a range of about 300-3000 nm, about 300-1500 nm, about 300-800 nm or about 400-700 nm.

35. Apparatus for a photodynamic therapy (PDT) treatment according to claim 1, wherein the first and second radiation sources operate in the visible region of the electromagnetic spectrum; and
wherein the first and second radiation sources operate in the ultraviolet or infrared wavelength regions of the electromagnetic spectrum.

36. Apparatus for a photodynamic therapy (PDT) treatment according to claim 1, wherein the first radiation source emits radiation in a range of between about 1 minute to three hours or between about 10 minutes to about 1 hour with a power of about 20-200 mW/cm²; and
wherein the first radiation source emits radiation in a constant or pulsed form.

37. Apparatus for a photodynamic therapy (PDT) treatment according to claim 1, wherein the photosensitiser is a photochemical and/or photopharmaceutical preparation such as Aminolevulinic Acid (ALA), Metvix, Methylene blue, Phenothiazinium compounds, Levulan; and
wherein the photosensitiser is in the form of a gel, ointment, cream or gauze soaked in a photodynamic therapy solution.

38. Apparatus for a photodynamic therapy (PDT) treatment according to claim 1, wherein the second radiation stage irradiates the surface of the patient to be treated in a second radiation stage over a longer period of time ranging from about six hours to about three months and preferably over a period of about 1 week to about 1 month wherein during this period the radiation is emitted in a continuous or non-continuous manner; and
wherein the radiation in the second radiation source is emitted in a non-constant manner and over a lower power than used in the first radiation stage and is carried out immediately or shortly following the first radiation stage such as about 0.1-20 mW/cm².

39. Apparatus for a photodynamic therapy (PDT) treatment according to claim 1, wherein the first and second radiation sources are any suitable form of diode such as organic light-emitting diode or an inorganic light-emitting diode incorporated into, for example, a diffuser; and
wherein the radiation sources are fluorescent light sources such as a fluorescent lamp.

40. Apparatus for a photodynamic therapy (PDT) treatment according to claim 1, wherein the first radiation source has an optical power density of about 20-200 mW/cm²; and
wherein the second radiation source has an optical power density of about 0.1-20 mW/cm².

41. Apparatus for a photodynamic therapy (PDT) treatment according to claim 1, wherein in the second radiation stage the photo sensitiser and illumination from the radiation source is administered simultaneously; and
wherein in the second radiation stage the photosensitiser and illumination from the radiation source is administered in sequence i.e. one after the other.

42. Apparatus for a photodynamic therapy (PDT) treatment according to claim 1, wherein the photosensitiser is applied at several times within the overall second irradiation stage such as once a day for a month; and
wherein the extended duration of the second irradiation stage requires an ambulatory device i.e. a mobile device to be worn by a person so that they no longer need to be kept as an in-house patient in a hospital.

43. Apparatus for a photodynamic therapy (PDT) treatment according to claim 1, wherein the photosensitiser and light radiation are applied simultaneously in at least one or both the first and second radiation stages which is capable of being achieved by a drug delivery mechanism which is capable of releasing photosensitiser in a controlled manner over the duration of the second stage; and
wherein the drug delivery mechanism is a transparent drug delivery layer or an iontophoretic drug delivery layer.

44. Apparatus for a photodynamic therapy (PDT) treatment according to claim 1, wherein the long duration of the second irradiation stage requires that a light source in the second radiation step is substantially off for most of the second stage; and
wherein the light source blips on for a short time such as for about 1 to 1,000 s.

45. Apparatus for a photodynamic therapy (PDT) treatment according to claim 1, wherein in the second radiation stage a low concentration of photosensitiser such as about 1-20% of that used in the first stage is used.

46. A method of providing a photodynamic therapy (PDT) treatment, said method comprising:
providing a first radiation source capable of irradiating the photosensitiser on the surface of the patient to be treated in a first radiation stage with a first wavelength over a short period of time ranging from about one minute to about three hours, wherein the photosensitiser is capable of performing medical treatment; and
providing a second radiation source capable of irradiating the surface of the patient to be treated in a second radiation stage with a second wavelength over a longer period of time ranging from about six hours to about three months.
